(19) United States
(12) Patent Application Publication
(10) Pub. No.: US 2002/0076426 A1
(43) Pub. Date: Jun. 20, 2002

(54) Use of terpene alcohol ethoxylates as solubilizers in cosmetic or pharmaceutical preparations or concentrates for food preparations

(76) Inventors: Michael Zirnstein, Schriesheim (DE); Tilman C. Rock, Sinsheim (DE); Karl Kolter, Limburgerhof (DE)

Correspondence Address: Herbert B. Keil, KEIL & WEINKAUF, 1101 Connecticut Ave., N.W., Washington, DC, 20036, US

(21) Appl. No.: 09/983,310

(22) Filed: Oct. 24, 2001

(30) Foreign Application Priority Data
Oct. 27, 2000 (DE) 10053512.7

(51) Int. Cl. A61K 47/30; A61K 7/00; A23L 1/00; A61K 47/32; A61K 47/34

(52) U.S. Cl. 424/401; 514/772.1; 426/531

(57) ABSTRACT

Use of terpene alcohol ethoxylates as solubilizers in cosmetic or pharmaceutical preparations or concentrates for food preparations

The use of terpene alcohols having 10 carbon atoms which have been ethoxylated with 3 to 10 mol of ethylene oxide as solubilizers for sparingly water-soluble or sparingly water-dispersible compounds in cosmetic or pharmaceutical preparations or in concentrates for food preparations, in amounts of at least 3% by weight, based on the finished preparations, or based on the concentrates.

The invention relates to the use of terpene alcohols which have been ethoxylated with 3 to 10 mol of ethylene oxide as solubilizers in cosmetic or pharmaceutical preparations or concentrates for food preparations, in amounts of at least 3% by weight.

From GB 2 297 557 it is known to be possible to make essential oils, e.g. geraniol, water-soluble by alkoxylation without considerable loss of the odor properties. The alkoxylates described therein are, accordingly, intended to be used subsequently as fragrances, i.e. in very small amounts, in preparations not described therein.

A large number of solubilizers from a very wide variety of compound groups are known for cosmetic and pharmaceutical preparations.

This is because in the preparation of homogeneous pharmaceutical or cosmetic preparations, the solubilization of hydrophobic substances has achieved very great practical importance.

Solubilization is to be understood as meaning an improvement in solubility brought about by surface-active compounds which are able to convert sparingly water-soluble or water-insoluble substances into clear, at most opalescent aqueous solutions, without the chemical structure of these substances being changed in the process.

The prepared solubilizates are notable for the fact that the sparingly water-soluble or water-insoluble substance is present in dissolved form in the molecular associates of the surface-active compounds which form in aqueous solution—“the micelles”. The resulting solutions are stable single-phase systems which appear optically clear to opalescent and can be prepared without the input of relatively large amounts of energy.

Solubilizers can improve the appearance of, for example, cosmetic formulations by making the formulations transparent. Furthermore, in the case of pharmaceutical preparations, the bioavailability and thus the action of medicinal substances can be increased through the use of solubilizers.

The solubilizers mainly used for pharmaceutical medicinal substances and cosmetic active ingredients are the following products:

ethoxylated (hydrogenated) castor oil, (e.g. Cremophor® grades, BASF);

ethoxylated sorbitan fatty acid esters, (e.g. Tween® grades, ICI);

ethoxylated hydroxystearic acid, (e.g. Solutol® grades, BASF).

Although these known solubilizers have proven successful, they are still in need of further improvement with regard to the solubilization and application properties, and also physiological compatibility.

It is an object of the present invention to propose novel solubilizers which combine very good solubilization properties, improved stability, good physiological compatibility and ready dosability in liquid form.

We have found that this object is achieved by reaction products of C₁₀-terpene alcohols with 3 to 10 mol of ethylene oxide, which are not only themselves readily soluble in water, but have an above-average solubilizing action for compounds which are sparingly soluble or sparingly dispersible in water, if they are used in amounts of at least 3% by weight, based on the finished preparations or concentrates for preparations.

Sparingly water-soluble or sparingly water-dispersible compounds, i.e. cosmetic or pharmaceutical active ingredients or food additives, are generally understood to mean compounds which have a solubility of less than 5% by weight and, in particular, of less than 1% by weight in water. Substances which are sparingly dispersible in water are usually substances which are insoluble or virtually insoluble in water which can only be converted into a stable dispersion in water or predominantly aqueous medium sing a solubilizer.

For pharmaceutical preparations, the term sparingly water-soluble active ingredients is here also intended to include the active ingredients according to DAB 9 (German pharmacopoeia) which are only slightly soluble in water. Here, the grading is as follows: only slightly soluble (soluble in 30 to 100 parts of solvent); sparingly soluble (soluble in 100 to 1000 parts of solvent); virtually insoluble (soluble in more than 10,000 parts of solvent).

The reaction products to be used according to the invention can also be readily dosed in liquid form, have greater resistance to hydrolysis than the esters customarily used and are physiologically compatible. In particular, they have very low haemolysis activity.

The compounds to be used according to the invention are, for example, nerol, linalool, menthol, terpineol and, in particular, geraniol and citronellol which have been reacted with 3 to 10 mol, preferably 5 to 7 mol, of ethylene oxide.

The solubilizers are used in amounts of from 3 to 90% by weight, preferably 3 to 50% by weight and in particular 3 to 30% by weight, based on the finished preparations or concentrates.

The preparation of the compounds to be used according to the invention by reaction with ethylene oxide is carried out in a manner known per se, as is described, for example, in N. Schönfeldt, Grenzflächenaktive Ethylenoxid-Addukte [Interface-active ethylene oxide adducts], Wissenschaftliche Verlagsgesellschaft mbH, Stuttgart, 1st Edition (1976) and supplementary volume (1984).

As a rule, the alcohol is reacted with ethylene oxide in the presence of a catalyst, preferably sodium hydroxide or potassium hydroxide (customarily 0.05 to 5% by weight, preferably 0.1 to 0.5% by weight, based on the alcohol to be oxethylated) to give the desired degree of oxethylation in an inert atmosphere. The catalyst may be added in the form of an aqueous solution. Prior to the actual metered addition of the ethylene oxide, the mixture is advantageously dewatered at 50 to 110° C., preferably at 60 to 90° C. and under reduced pressure. The oxethylation of primary alcohols is carried out at 90 to 150° C., preferably at 120 to 140° C.; the oxethylation of secondary alcohols is carried out at 120 to 170° C., preferably at 140 to 160° C.

The reaction product may optionally be subjected to an after treatment. Such treatments include, inter alia, the neutralization of the catalyst by the addition of acids, such as, for example, phosphoric acid, or ion exchangers or the addition of adsorbents such as silicates or aluminum oxides. The after treatment can be carried out at temperatures of from 10 to 180° C., preferably 30 to 100° C., and takes place with stirring over 0.5 to 12 h. This is usually followed by a filtration step, optionally using a filtration auxiliary, such as, for example, diatomaceous earth. A combination of these agents may also be useful for the after treatment.

The silicates are preferably alkali metal or alkaline earth metal silicates, in particular Mg silicates or Ca silicates, or A1 silicates, but also bleaching earths. Commercial products which may be mentioned are, for example, Ambosol, Tonsil FF or Magnesol.

### Applications

The present invention provides water-soluble, amphiphilic compounds for use as solubility promoters for pharmaceutical and cosmetic preparations. They have the property of solubilizing sparingly soluble active ingredients in the field of pharmacy and cosmetics, sparingly soluble food supplements, for example vitamins and carotinoids, but also sparingly soluble active ingredients for use in crop protection compositions, and active ingredients in aqueous systems used in veterinary medicine.

### Solubilizers for Cosmetics

The compounds to be used according to the invention may be used as solubilizers in cosmetic formulations. They are particularly suitable as solubilizers for cosmetic oils. They have good solubilizing ability for fats and oils, such as groundnut oil, jojoba oil, coconut oil, almond oil, olive oil, palm oil, castor oil, soybean oil or wheatgerm oil, or for essential oils, such as dwarf-pine oil, lavender oil, rosemary oil, spruce needle oil, pine needle oil, eucalyptus oil, peppermint oil, sage oil, bergamot oil, turpentine oil, melissa oil, juniper oil, lemon oil, aniseed oil, cardamom oil, camphor oil etc. or for mixtures of these oils.

In addition, the compounds to be used according to the invention can be used as solubilizers for UV absorbers which are insoluble or sparingly soluble in water, such as, for example, 2-hydroxy-4-methoxybenzophenone (Uvinul® M 40, BASF), 2,2′,4,4′-tetrahydroxybenzophenone (Uvinul® D 50), 2,2′-dihydroxy-4,4′-dimethoxybenzophenone (Uvinul® D49), 2,4-dihydroxybenzophenone (Uvinul® 400), 2-ethylhexyl 2-cyano-3,3-diphenylacrylate (Uvinule® N 539), 2,4,6-trianilino-p-(carbo-2′-ethylhexyl-1′-oxy)-1,3,5-triazine (Uvinul® T 150), 3-(4-methoxybenzylidene)camphor (Eusolex® 6300, Merck), 2-ethylhexyl N,N-dimethyl-4-aminobenzoate (Eusolex® 6007), 3,3,5-trimethylcyclohexyl salicylate, 4-isopropyldibenzoylmethane (Eusolex® 8020), 2-ethylhexyl p-methoxycinnamate and 2-isoamyl p-methoxycinnamate, and mixtures thereof.

The present invention therefore also provides cosmetic preparations which comprise at least one of the compounds to be used according to the invention as solubilizers. Preference is given to preparations which, in addition to the solubilizer, comprise one or more sparingly soluble cosmetic active ingredients, for example the aforementioned oils or UV absorbers.

These formulations are solubilizates based on water or water/alcohol. The compound I is used as solubilizer in the ratio of from 0.2:1 to 50:1, preferably 0.5:1 to 20:1, particularly preferably 1:1 to 15:1, very particularly preferably 2:1 to 12:1 to the sparingly soluble cosmetic active ingredient.

Additionally, further auxiliaries may be added to this formulation, for example nonionic, cationic or anionic surfactants, such as alkyl polyglycosides, fatty alcohol sulfates, fatty alcohol sulfonates, fatty alcohol ether sulfates, fatty alcohol ether sulfonates, alkanesulfonates, fatty alcohol ethoxylates, fatty alcohol phosphates, alkylbetaines, sorbitan esters, POE sorbitan esters, sugar fatty acid esters, fatty acid polyglycerol esters, fatty acid partial glycerides, fatty acid carboxylates, fatty alcohol sulfosuccinates, fatty acid sarcosinates, fatty acid isethionates, fatty acid taurates citric esters, silicone copolymers, fatty acid polyglycol esters, fatty acid amides, fatty acid alkanolamides, quaternary ammonium compounds, alkylphenol oxethylates, fatty amine oxethylates, cosolvents, such as ethylene glycol, propylene glycol, glycerol etc.

Further constituents which may be added are natural or synthetic compounds, e.g. lanolin derivatives, cholesterol derivatives, isopropyl myristate, isopropyl palmitate, electrolytes, dyes, preservatives, acids (e.g. lactic acid, citric acid).

These formulations are used, for example, in bath preparations such as bath oils, aftershaves, face washes, mouth rinses, hair tonics, eau de cologne, eau de toilette etc.

### Description of the Solubilization Method

In the preparation of the solubilizates for cosmetic formulations, the compounds to be used according to the invention can be used as 100% strength substance or as aqueous solution.

Usually, the solubilizer is dissolved in water and vigorously mixed with the sparingly soluble cosmetic active ingredient to be used in each case, e.g. the aforementioned essential oils or perfume oils, for example using a magnetic stirrer.

It is, however, also possible to dissolve the sparingly soluble cosmetic active ingredient to be used in a melt of the solubilizer and then to add demineralized water with continuous stirring.

### Solubilizers for Pharmaceutical Applications

The compounds to be used according to the invention are suitable for use as solubilizers in pharmaceutical preparations of any type which are characterized in that they comprise one or more sparingly water-soluble or water-insoluble medicinal substances or vitamins, and carotinoids. In particular, these are aqueous solutions or solubilizates for oral or parenteral application, such as, for example, injection solutions for intravenous, intramuscular or subcutaneous or intraperitoneal application.

Furthermore, the compounds to be used according to the invention are suitable for use in oral administration forms, such as tablets, capsules, powders, solutions. Here, they are able to make available the sparingly soluble medicinal substance with increased bioavailability.

In the case of parenteral application, as well as using solubilizers, it is also possible to use emulsions, for example fatty emulsions. For this purpose too, the claimed compounds are suitable for processing a sparingly soluble medicinal substance.

Pharmaceutical formulations of the aforementioned type can be obtained by processing the claimed compounds with pharmaceutical active ingredients by traditional methods and using known and novel active ingredients.

The use according to the invention can additionally include pharmaceutical auxiliaries and/or diluents. Specific auxiliaries are cosolvents, stabilizers and preservatives.

The active ingredients may be from any indication area. Examples which may be mentioned here are benzodiazepines, antihypertensives, vitamins, cytostatics, in particular taxol, anesthetics, neuroleptics, antidepressants, antibiotics, antimycotics, fungicides, chemotherapeutics, urologics, thrombocyte aggregation inhibitors, sulfonamides, spasmolytics, hormones, immunoglobulins, sera, thyroid therapeutic agents, psychopharmacological agents, agents for treating Parkinson's disease and other antihyperkinetic agents, ophthalmics, neuropathy preparations, calcium metabolic regulators, muscle relaxants, narcotics, antilipemics, hepatic therapeutic agents, coronary agents, cardiacs, immunotherapeutics, regulatory peptides and their inhibitors, hypnotics, sedatives, gynecological agents, gout remedies, fibrinolytics, enzyme preparations and transport proteins, enzyme inhibitors, emetics, circulation-promoting agents, diuretics, diagnostics, corticoids, cholinergics, bile duct therapeutics, antiasthmatics, broncholytics, beta receptor blockers, calcium antagonists, ACE inhibitors, arteriosclerotics, antiphlogistics, anticoagulants, antihypotonics, antihypoglycemics, antihypertonics, antifibrinolytics, antiepileptics, antiemetics, antidotes, antidiabetics, antiarrhythmics, antianemics, antiallergics, anthelmintics, analgesics, analeptics, aldosterone antagonists and slimming agents.

The compounds according to the invention are used as solubility promoters in pharmaceutical preparations by, for example, dispersing or dissolving the active ingredient in the solubilizer, optionally with heating, and mixing it with water with stirring.

Another preparation variant involves dissolving the solubilizer in the aqueous phase, optionally with gentle warming, and subsequently dissolving the active ingredient in the aqueous solubilizer solution. The simultaneous dissolution of solubilizer and active ingredient in the aqueous phase is likewise possible.

The invention thus also provides pharmaceutical preparations which comprise at least one of the compounds to be used according to the invention as solubilizers. Preference is given to preparations which, in addition to the solubilizer, comprise a sparingly water-soluble or water-insoluble pharmaceutical active ingredient, for example from the aforementioned indication areas.

Of the aforementioned pharmaceutical preparations, particular preference is given to those which are formulations which can be administered parenterally.

The content of solubilizer according to the invention in the pharmaceutical preparation is, depending on the active ingredient, in the range from 3 to 90% by weight, preferably 3 to 50% by weight, particularly preferably 3 to 30% by weight.

### Solubilizers for Food Preparations

As well as the use in cosmetics and pharmaceuticals, the compounds to be used according to the invention are also suitable as solubilizers in the food sector for sparingly water-soluble or water-insoluble nutrients, supplements or additives, such as, for example, fat-soluble vitamins or carotinoids. Examples which may be mentioned are clear drinks colored with carotinoids.

The invention therefore also provides concentrates (masterbatches) for food preparations where the concentrates comprise at least one of the compounds to be used according to the invention as solubilizers in amounts of at least 3% by weight. Preference is given to concentrates which, in addition to the solubilizer, comprise a sparingly water-soluble or water-insoluble vitamin or carotinoid.

### EXAMPLES

### Preparation of the Ethoxylates

### Example 1

### Citronellol+8 EO

A mixture of 265.5 g (1.70 mol) of citronellol and 0.53 g of potassium hydroxide was dewatered at 70° C. under reduced pressure (<10 mbar) for 1 h. 600 g (13.6 mol) of ethylene oxide were metered into an autoclave rendered inert with nitrogen, with stirring at 140° C. over the course of 3.5 h. During this operation, a gage pressure of up to 7 bar was achieved. After the ethylene oxide metered addition was complete, the mixture was stirred for a further 1 h at 140° C. The mixture was stirred for a further hour at 70° C. and at a reduced pressure of <10 mbar, treated with 3.54 g of Ambosol® (Hoechst), stirred for one hour at 80° C. and pressure-filtered. Yield: 850 g (98.2%), OH number=109 mg of KOH/g.

### Example 2

### Citronellol+5.5 EO

A mixture of 330 g (2.12 mol) of citronellol and 0.66 g of potassium hydroxide was dewatered at 60 to 70° C. under reduced pressure (≧1 mbar) to a residual water content of 0.04%. In an autoclave rendered inert with nitrogen, 471 g (10.7 mol) of ethylene oxide were metered into 312 g (2.00 mol) of said citronellol mixture over the course of 4 h, with stirring at 130° C. During this operation, a gage pressure of up to 7 bar was reached. When the ethylene oxide metered addition was complete, the mixture was stirred for a further 1 h at 130° C. The mixture was stirred for a further hour at 70° C. and at a reduced pressure of ≧1 mbar, treated with 4.40 g of Ambosol, stirred for one hour at 80° C. and pressure-filtered. Yield: 772 g (98.6%), OH number=142 mg of KOH/g.

### Example 3

### Geraniol+9 EO

A mixture of 277.6 g (1.80 mol) of citronellol and 0.56 g of potassium hydroxide was dewatered at 70° C. under reduced pressure (<10 mbar) for 1 h. 717 g (16.3 mol) of ethylene oxide were metered into an autoclave which had been rendered inert with nitrogen, with stirring at 140° C. over the course of 4.5 h. During this operation, a gage pressure of up to 7 bar was achieved. After the ethylene oxide metered addition was complete, the mixture was stirred for a further 1.5 h at 140° C. The mixture was stirred for a further hour at 70° C. and a reduced pressure of <10 mbar, treated with 3.31 g of Ambosol, stirred for one hour at 70° C. and pressure-filtered. Yield: 1005 g (quant.), OH number=100 mg of KOH/g.

### Use

### Example 4

### Piroxicam Injection Solution

2.5 g of Piroxicam were introduced, with stirring, into a mixture of 200 g of citronellol ethoxylate having 9 mol of ethylene oxide and 2.0 g of benzyl alcohol. The mixture was then heated to 50° C. and stirred until the Piroxicam had completely dissolved. 795.4 g of water for injection purposes were then slowly added to the clear solution, and the mixture was cooled to room temperature.

Following sterilization by filtration using a 0.22 μm filter, the clear solution was transferred under sterile conditions to 10 ml ampoules, and the ampoules were sealed.

### Example 5

### Vitamin E Acetate Drinking Solution

25 g of vitamin E acetate were dissolved, with stirring at 60° C., in 200 g of citronellol ethoxylate having 6 mol of ethylene oxide. 775 g of a separately prepared solution of 200 g of sorbitol, 1.0 g of aspartame and 2.0 g of mandarin flavoring in 572.0 g of purified water was then slowly added to this solution. After cooling to room temperature, the vitamin E acetate drinking solution was transferred to 100 ml bottles with screw-cap closure.

### Example 6

### Diazepam Granulate

10.0 g of diazepam were dissolved at 50° C. in 200.0 g of a mixture of 6 parts by weight of geraniol ethoxylate having 7 mol of ethylene oxide and 4 parts by weight of ethanol. This solution was sprayed onto a powder mixture consisting of 1000.0 g of lactose, 850.0 g of microcrystalline cellulose (Avicel® PH 102) and 20.0 g of highly disperse silica (Aerosil® OX 50) in a Lödige plowshare mixer. The granulate was then dried off in a tray drying cabinet at 30° C. and transferred to 2.0 g sachets.

### Example 7

### Nifedipine Capsules

40.0 g of nifedipine were dissolved, with the exclusion of light and with stirring, in a mixture of 200.0 g of citronellol ethoxylate having 6 mol of ethylene oxide, 400.0 g of polyethylene glycol 6000 and 360.0 g of polyoxyethylene/polyoxypropylene block polymer (Lutrol®, F 68) at 60° C. This solution was transferred to hard gelatin capsules of size 00, 500 mg being metered in in each case. After the capsule contents had cooled, the upper and lower parts of the capsule were tightly sealed by banding with 30% strength gelatin solution.

### Examples 8 to 13 (Comparative Examples 8 to 10)

The terpene alcohol ethoxylates were each heated to 65° C. and added to the medicinal substance. The phosphate buffer pH 7.0 (USP XXIII; 34.0 g of potassium dihydrogenphosphate and 145.5 ml of 1N sodium hydroxide solution dissolved in 4000.0 g of demineralized water and made up to 5.0 1 with demineralized water) was then added in small portions until the solubilizer concentration was 20% by weight, based on the finished solution. The mixture was stirred at room temperature until the saturation concentration of the medicinal substance had been reached. The resulting saturation concentrations in % by weight are given in the table below.

TABLE

| | | Sulfathia- | | |
|---|---|---|---|---|
| Ex. | Compound | zole | 17-β-Estradiol | Clotrimazole |
| 8 | Phosphate buffer | 0.07 | 0.0 | 0.0 |
| | pH 7.0 alone | | | |
| 9 | Sorbitan fatty | 0.7 | 0.09 | 0.03 |
| | acid ester | | | |
| | (Tween ® 80) | | | |
| 10 | Ethoxylated | 0.7 | 0.06 | 0.01 |
| | castor oil | | | |
| | (Cremophor ® EL) | | | |
| 11 | Citronellol | 0.95 | 0.23 | 0.39 |
| | ethoxylate with | | | |
| | 8 EO | | | |
| 12 | Citronellol | 0.78 | 0.30 | — |
| | ethoxylate with | | | |
| | 6 EO | | | |
| 13 | Citronellol | — | 0.14 | — |
| | ethoxylate with | | | |
| | 9 EO | | | |

### Example 14

### Sunscreen Composition

25 g of citronellol ethoxylate with 6 EO were heated to 60° C., and 2.5 g of Uvinul® T 150 were dissolved therein with stirring. Then, a mixture of 62.5 g of double-distilled water, 5 g of glycerol and 5 g of 1,2-propylene glycol, heated to 60° C., was carefully added dropwise with stirring. This gave a clear solution which, after cooling to room temperature, was transferred to a suitable container.

We claim:

1. The use of terpene alcohols having 10 carbon atoms which have been ethoxylated with 3 to 10 mol of ethylene oxide as solubilizers for sparingly water-soluble or sparingly water-dispersible compounds in cosmetic or pharmaceutical preparations or concentrates for food preparations, in amounts of at least 3% by weight, based on the finished cosmetic and pharmaceutical preparations, or based on the concentrates for food preparations.

2. The use as claimed in claim 1, wherein the C₁₀-terpene alcohol ethoxylate is citronellol reacted with 5 to 7 mol of ethylene oxide.

3. The use as claimed in claim 1, wherein the C₁₀-terpene alcohol ethoxylate is geraniol reacted with 5 to 7 mol of ethylene oxide.

4. A cosmetic or pharmaceutical preparation comprising at least 3% by weight of C₁₀-terpene alcohols alkoxylated with 3 to 10 mol of ethylene oxide as solubilizers for sparingly water-soluble or sparingly water-dispersible active ingredients.

5. A cosmetic or pharmaceutical preparation as claimed in claim 4, comprising 3 to 50% by weight, based on the finished preparations, of C₁₀-terpene alcohols alkoxylated with 3 to 10 mol of ethylene oxide as solubilizers for sparingly water-soluble or sparingly water-dispersible active ingredients.

6. A cosmetic or pharmaceutical preparation as claimed in claim 4, wherein the C₁₀-terpene alcohol ethoxylate is citronellol reacted with 5 to 7 mol of ethylene oxide.

7. A cosmetic or pharmaceutical preparation as claimed in claim 4, wherein the C₁₀-terpene alcohol ethoxylate is geraniol reacted with 5 to 7 mol of ethylene oxide.

8. A concentrate for food preparations, comprising 3 to 50% by weight of C₁₀-terpene alcohols alkoxylated with 3 to 10 mol of ethylene oxide as solubilizers for sparingly water-soluble or sparingly water-dispersible food additives.
